(19) United States
(12) Patent Application Publication
(10) Pub. No.: US 2003/0091528 A1
(43) Pub. Date: May 15, 2003

(54) Transparent liquid composition

(76) Inventors: Masashi Goto, Hyogo (JP); Akio Maekawa, Hyogo (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/258,430

(22) PCT Filed: Apr. 24, 2001
(86) PCT No.: PCT/JP01/03540

(30) Foreign Application Priority Data
Apr. 24, 2000 (JP) 2000-122114

(51) Int. Cl. A61K 7/075; A61K 7/08

(52) U.S. Cl. 424/70.31

(57) ABSTRACT

A transparent liquid composition comprising water, an oil-soluble ingredient and a surfactant; more specifically, a transparent liquid composition comprises 50 wt % or more of water, 30 wt % or lower of an oil-soluble ingredient and a 20 wt % or lower of a surfactant wherein the content of the surfactant is less than that of the oil-soluble ingredient.

### TECHNICAL FIELD

The present invention relates to a transparent liquid composition.

### BACKGROUND ART

Compositions comprising an oil-soluble ingredient dissolved and/or finely dispersed in water have been widely used in the fields of pharmaceuticals, quasi-drugs, cosmetics, oral products, foods and the like. For example, transparent infusions or injections prepared by dissolving and/or finely dispersing an oil-soluble nutriment ingredient such as an oil-soluble vitamin in water using a large amount of a surfactant are used in the pharmaceutical field. However, if a infusion or injection containing a large amount of a surfactant is injected, a patient might manifest a shock symptom. Among infusions, a fat emulsion prepared by emulsifying and dispersing a soybean oil or the like, may pose a problem that with time or in mixing with other infusion, fat particles are increased in diameter, thereby reportedly causing a pulmonary fat embolism syndrome. In the fields of quasi-drugs, cosmetics and oral products, compositions having a transparent appearance for increasing the stability or for making the appearance more attractive are produced by use of a surfactant highly capable of solubilizing an oil-soluble ingredient or by use of a large amount of a surfactant. Although depending on the kind of surfactant, the composition usually contains a surfactant in a larger amount than an oil-soluble ingredient to obtain a transparent composition by solubilizing the oil-soluble ingredient. However, such transparent compositions have problems. In such transparent composition, the effect of oil-soluble ingredient is lowered by the surfactant or the irritation to skin is increased. In the food field, transparent compositions are provided from viewpoints of stability and appearance, but entail a problem of impairing the flavor due to a large amount of surfactant used.

Also proposed was a composition which is characterized by containing ethanol or like solvent in a larger amount than water for dissolving an oil-soluble ingredient as well as by using a surfactant having a high solubilizing capabilty or using a large amount of a surfactant. However, such compositions have problems. The proposed compositions are limited to those comprising an oil-soluble ingredient which is soluble in ethanol. Further, a large amount of ethanol used increases the irritation to skin. Japanese Unexamined Patent Publications No. 183,608/1983 and No. 206,509/1983 disclose compositions which look transparent due to the adjustment of refractive indexes of oil-soluble ingredient to aqueous ingredient by adding polyhydric alcohol having 3 or more OH groups. The compositions which look transparent due to the presence of polyhydric alcohol, however, do not overcome the problem of the stability. Further proposals include a composition in which an oil-soluble ingredient is solubilized to become transparent by combined use of a polyhydric alcohol and an aliphatic higher alcohol which is a liquid at room temperature (Japanese Patent Publication No. 2,575,737) and a transparent composition comprising a polyglycerin polyoxybutylene alkylether compound and an unsaturated aliphatic alcohol having 14 to 24 carbon atoms (Japanese Patent Publication No. 2,724,852). However, these compositions raise a problem that the effect of the oil-soluble ingredient is lowered due to the ingredient used in combination.

### DISCLOSURE OF THE INVENTION

An object of the invention is to provide a transparent liquid composition which overcomes the foregoing drawbacks.

The present inventor found a composition comprising water, an oil-soluble ingredient and a surfactant, the composition being free of the foregoing drawbacks, and completed this invention.

That is, the invention provides what is described in the following items.

Item 1. A transparent liquid composition comprising water, an oil-soluble ingredient and a surfactant.

Item 2. The transparent liquid composition according to item 1, which comprises 50 wt % or more of water, 30 wt % or lower of an oil-soluble ingredient and a 20 wt % or lower of a surfactant, the composition being characterized in that the content of the surfactant is less than the content of the oil-soluble ingredient.

Item 3. The transparent liquid composition according to item 1, which is characterized in that the content of the surfactant is ½ or lower the content of the oil-soluble ingredient.

Item 4. The transparent liquid composition according to item 1, which is characterized in that the surfactant is one having an HLB value of 12 or more.

Item 5. The transparent liquid composition according to item 1, which is characterized in that the surfactant is nonionic surfactant.

Item 6. The transparent liquid composition according to item 1, which is characterized in that the composition has a transmission of 70% or more.

Item 7. A process for preparing the transparent liquid composition of item 1, the process comprising the steps of mixing water, an oil-soluble ingredient and a surfactant to obtain an opaque mixture and further mixing the opaque mixture under an increased pressure.

The transparent liquid composition of the invention comprises as the essential ingredients water, an oil-soluble ingredient and a surfactant.

The term “transparent” used herein refers to a case wherein the transmission of composition is in the range of about 70% or more, preferably about 80% or more. The transmission can be measured in terms of wavelength in the region of visible light rays with a spectrophotometer.

The term “oil-soluble ingredient” used herein means an ingredient which is substantially insoluble in water at room temperature.

The oil-soluble ingredient to be incorporated in the composition of the invention is not limited and can be suitably selected from pharmaceutically effective ingredients, active ingredients, fragrance, vitamins, vegetable oils, hydrocarbons, fatty acid esters, silicons, fatty acids, hormones and like useful ingredients which are usually used in the fields of pharmaceuticals, quasi-drugs, cosmetics, oral products, foods and the like. The composition of the invention can contain at least one kind of oil-soluble ingredient. Useful oil-soluble ingredients are, for example, triclosan, isopropylmethylphenol and like bactericides; menthol, spearmint oil, peppermint oil, lemon oil, orange oil and like aromas; tocopherol acetate, tocopherol succinate, tocopherol nicotinate and like vitamins E; carotin, retinol and like vitamins A; olive oil, jojoba oil, soybean oil and like vegetable oils; liquid paraffin, squalane and like hydrocarbons; isopropyl palmitate, ethyl oleate, cetyl octanoate, octyl stearate, octyldodecyl lactate and like fatty acid esters; dimethylsilicon, cyclic silicon and like silicons; oleic acid, linoleic acid and like fatty acids; ethynylestradiol and like hormones.

The oil-soluble ingredient to be used in the invention is preferably one which is a liquid (including an oil or paste) at room temperature, and includes one which is a solid or powder at room temperature. When using the oil-soluble ingredient which is a solid or powder at room temperature, the ingredient is usually heated to form a liquid or is dissolved in other oil-soluble ingredient which is a liquid at room temperature.

The kind of the surfactant to be used in the composition of the invention can be suitably selected according to the kind of the oil-soluble ingredient from surfactants generally used for pharmaceuticals, quasi-drugs, cosmetics, oral products, foods and the like.

The surfactant to be used has an HLB value of about 12 or more, preferably about 13 or more.

Specific examples of preferred surfactants are nonionic surfactants; chlorinated stearyltrimethylammonium, chlorinated distearyldimethylammonium and like cationic surfactants; sodium lauryl sulfate, sodium lauroyl methylalanine, sodium N-cocoylmethyltaurine and like anionic surfactants; coconut oil fatty acid amidopropyl dimethylaminoacetate betaine, 2-alkyl-N-carboxymethyl-N-hydroxyethyl imidazolinium betaine and like amphoteric surfactants; amidoamine or acid neutralization products thereof; and lecithin.

Useful amidoamines are, for example, stearic acid dimethylaminopropylamide, stearic acid diethylaminoethylamide and the like. Neutralization products of amidoamine to be used include, for example, neutralization products prepared by neutralizing amidoamine with an acid such as phosphoric acid, citric acid, glutamic acid, hydrochloric acid or the like.

Lecithins to be used include, for example, yolk lecithin, hydrogenated yolk lecithin, yolk lysolecithin, hydrogenated yolk lysolecithin, soybean lecithin, hydrogenated soybean lecithin, soybean lysolecithin, hydrogenated soybean lysolecithin, etc.

Among them, preferred surfactants to be incorporated in the composition of the invention include, for example, nonionic surfactants.

Examples of the nonionic surfactant are polyoxyethylene alkylether, polyoxyethylene polyoxypropylene alkylether, polyoxyethylene fatty acid ester, polyoxyethylene sorbitan fatty acid ester, polyoxyethylene sorbit fatty acid ester, polyoxyethylene hardened castor oil, polyoxyethylene alkylphenylether, polyglycerin fatty acid ester, etc.

A preferred polyoxyethylene alkylether is such that the polymerization degree of ethylene oxide is about 4 to about 40, and the alkyl group has about 12 to about 22 carbon atoms. Specific examples are polyoxyethylene (9) lauryl ether (HLB=14.5), polyoxyethylene (15) oleyl ether (HLB=16.0), polyoxyethylene (20) oleyl ether (HLB=17.0), polyoxyethylene (20) stearyl ether (HLB=18.0), polyoxyethylene (30) behenyl ether (HLB=18.0) and the like.

A preferred polyoxyethylene polyoxy-propylene alkylether is such that the polymerization degree of ethylene oxide is about 4 to about 40, the polymerization degree of propylene oxide is about 1 to about 20 and the alkyl group has about 12 to about 30 carbon atoms. Specific examples are polyoxyethylene (20) polyoxypropylene (1) cetyl ether (HLB=16.0), etc.

A preferred polyoxyethylene fatty acid ester is such that the polymerization degree of ethylene oxide is about 8 to about 60, and the fatty acid has about 12 to about 22 carbon atoms. Specific examples are polyoxyethylene (25) monooleate (HLB=15.0), polyoxyethylene (40) monostearate (HLB=17.5), etc.

A preferred polyoxyethylene sorbitan fatty acid ester is such that the polymerization degree of ethylene oxide is about 6 to about 30, and the fatty acid has about 12 to about 22 carbon atoms. Specific examples are polyoxyethylene (20) sorbitan monolaurate (HLB=16.9), polyoxyethylene (20) sorbitan monooleate (HLB=15.0), etc.

A preferred polyoxyethylene sorbitol fatty acid ester is such that the polymerization degree of ethylene oxide is about 4 to about 100, and the fatty acid has about 12 to about 22 carbon atoms. Specific examples are polyoxyethylene (60) sorbitol tetraoleate (HLB=14.0), etc.

A preferred polyoxyethylene hardened castor oil is such that the polymerization degree of ethylene oxide is about 30 to about 200. Specific examples are polyoxyethylene (60) hardened castor oil (HLB=14.0), polyoxyethylene (100) hardened castor oil (HLB=16.5), etc.

A preferred polyoxyethylene alkylphenylether is such that the polymerization degree of ethylene oxide is about 6 to about 50, and the alkyl group has about 6 to about 20 carbon atoms. Specific examples are polyoxyethylene (30) octylphenylether (HLB=20.0), polyoxyethylene (10) nonylphenylether (HLB=16.5), etc.

A preferred polyglycerin fatty acid ester is such that the polymerization degree of glycerin is about 6 to about 12, and the fatty acid has about 12 to about 25 carbon atoms. Specific examples are hexaglyceryl monolaurate (HLB=14.5), decaglyceryl monooleate (HLB=12.0), decaglyceryl monolaurate (HLB=15.5), etc.

These surfactants can be used either alone or in combination.

The proportions of water, surfactant and oil-soluble ingredient in the liquid composition of the invention are not limited. Usually the composition comprises preferably about 50 wt % or more of water, about 30 wt % or less of surfactant, about 20 wt % or less of oil-soluble ingredient, more preferably about 75 wt % or more of water, about 15 wt % or less of oil-soluble ingredient and about 10 wt % or less of surfactant, most preferably about 90 wt % or more of water, about 7 wt % or less of oil-soluble ingredient and about 3 wt % or less of surfactant, based on the total weight of the composition.

The upper limit of the amount of water is not limited but usually about 99.899 wt % in which case the lower limit of the amount of oil-soluble ingredient is about 0.1 wt % and that of surfactant is 0.001 wt %.

The amount of the surfactant is preferably smaller than the oil-soluble ingredient in terms of weight, preferably about ½ or less, more preferably about ⅓ or less, most preferably about ⅕ or less, especially preferably about 1/10 or less, that of the oil-soluble ingredient. When the amount of the surfactant meets the foregoing requirement, the surfactant is more unlikely to impair the effect of oil-soluble ingredient. Hence it is preferred. The lower limit of ratio of surfactant to oil-soluble ingredient is not limited, but usually about 1/1000.

The transparent liquid composition of the invention can be used in the fields of pharmaceuticals, quasi-drugs, cosmetics, oral products, foods and the like. Consequently the transparent liquid composition of the invention may contain other ingredients than oil-soluble useful ingredients insofar as they are in the range wherein the contemplated effect of the invention are achieved. Other ingredients to be added can be suitably selected according to the utility from water-soluble humectants, diluting agents, high molecular weight substances, antioxidants, UV absorbing agents, antiseptics, chelating agents, amino acids, water-soluble vitamins, coloring agents, pH-adjusting agents such as organic or inorganic acids, and the like which are usually used in the fields of pharmaceuticals, quasi-drugs, cosmetics, orally applicable products, foods and the like.

Examples of these ingredients are humectants such as polyethylene glycol, glycerin, 1,3-butylene glycol, propylene glycol, dipropylene glycol, sodium pyrrolidone carboxylate and the like; diluting agents such as ethyl alcohol, isopropyl alcohol and the like; high molecular weight substances such as hydroxyethyl cellulose, cationic cellulose, polyethylene glycol, protein derivatives, resins and the like; antiseptic agents such as paraben; chelating agents such as sodium ethylenediamine tetraacetate, sodium 1-hydroxyethane-1,1-diphosphonate and the like; amino acids such as glutamic acid, aspartic acid and the like; water-soluble vitamins such as ascorbic acid, vitamin B group and the like; coloring agents such as tar-dyes; pH adjusting agents such as citric acid, glycolic acid, phosphoric acid, hydrochloric acid, sodium citrate, sodium glycolate, sodium phosphate, sodium hydroxide and the like.

The composition of the invention can be obtained, for example, by mixing water, an oil-soluble ingredient, a surfactant and optionally other ingredients to obtain an opaque mixture and further mixing the opaque mixture under an increased pressure and under specific conditions which increases the transparency of the mixture.

The method of preliminarily mixing (“premixing”) the ingredients is not limited, and can be carried out with use of a high-speed stirrer, supersonic homogenizer or the like conventionally employed. The conditions for premixing are not limited. The premixing can be conducted under either an increased pressure or the atmospheric pressure. Prior to premixing, the oil-soluble ingredient which is a solid or powder at room temperature may be heated to form a liquid or may be dissolved in other oil-soluble ingredient which is a liquid at room temperature.

When a high-speed stirrer is used in premixing, the mixture may be stirred at about 1,000 to about 20,000 rpm for about 3 to about 60 minutes.

When a supersonic homogenizer is used in premixing, each ingredient is supplied at a specified flow rate to a supersonic irradiation chamber and the mixture is dispersed at an oscillating frequency of about 20 kHz by supersonic irradiation. Thereby an opaque mixture can be produced.

The degree of premixing is not limited. As each ingredient is more finely dispersed in the obtained opaque mixture, a more transparent liquid composition is obtained by mixing under an increased pressure to be described later.

The opaque mixture obtained by premixing is further mixed under an increased pressure to become transparent. The mixing is effected under an increased pressure using, for example, a high pressure homogenizer, a fluid mixing device (ramond mixer) disclosed in Japanese Examined Patent Publication No. 39,173/1984, or a device in which the mixture is passed through a microporous membrane as disclosed in Japanese Examined Patent Publication No. 2,416/1996.

The conditions for imparting transparency to the opaque mixture with a high pressure homogenizer are not limited insofar as a transparent composition is obtained under such conditions. Suitable conditions can be properly selected according to the kinds of surfactant and oil-soluble ingredient. For example, the mixture is mixed under a pressure of about 10 to about 200 MPa, preferably about 50 to about 150 MPa at room temperature or optionally while being heated, or is circulated 2 or 5 times before mixing when so required.

When the ramond mixer is used, the mixing can be done under a pressure of about 0.1 to about 10 MPa at room temperature or optionally at an elevated temperature with the device, for example, described in Japanese Examined Patent Publication No. 39,173/1984. When required, the mixture may be circulated 2 to 5 times before mixing.

When the opaque mixture is further mixed using a microporous membrane, the opaque mixture obtained by premixing is passed through a microporous membrane by applying a pressure under which a transparent composition can be obtained, usually a pressure of about 0.3 to about 20 MPa. Optionally the mixture may be circulated 2 to 5 times before passing through a microporous membrane. The microporous membrane may be made of organic or inorganic material insofar as it has a uniform pore diameter. Examples of such membranes are those made of, for example, porous glass prepared from CaO—B₂O₃—SiO₂—Al₂O₃ disclosed in Japanese Examined Patent Publication No. 25,618/1987, porous glass prepared from CaO—B₂O₃—SiO₂—Al₂O₃—Na₂O and porous glass prepared from CaO—B₂O₃—SiO₂—Al₂O₃—Na₂O—MgO disclosed in Japanese Unexamined Patent Publication No. 40,841/1986 and in the specification of U.S. Pat. No. 4,657,875.

The term “uniform pore diameter” refers to the following pore diameter: when a pore diameter of microporous membrane (wherein the pore volume occupies 10% of the total) is divided by a pore diameter thereof (wherein the pore volume occupies 90% of the total) in a relative cumulative pore distribution curve, the obtained value is in the range of approximately 1 to 1.5 as disclosed in Japanese Examined Patent Publication No. 2,146/1996.

An average pore diameter of the microporous membrane to be used for imparting transparency to the opaque mixture can be suitably selected according to the transparency to be attained, and is usually about 1 μm or less, preferably about 0.8 μm or less, more preferably about 0.5 μm or less.

The thickness of the microporous membrane is not limited but usually about 0.4 to about 2 mm.

Among these exemplified producing processes, a preferred process for preparing the transparent liquid composition of the invention comprises mixing the mixture under an increased pressure using a microporous membrane.

The above-described process is merely an example of processes for preparing the transparent liquid composition of the invention. The transparent liquid compositions of the invention are not limited to those obtained by the above-exemplified process under the above-mentioned conditions.

The composition of the invention, if required to be transparent, is usable in limitless fields which include pharmaceuticals, quasi-drugs, cosmetics, oral products, foods and the like. For example, among pharmaceutical infusions, the fat emulsion is an opaque liquid. However, when the invention is applied, a transparent liquid fat preparation can be produced. Further the transparent liquid composition of the invention is employable as an object filled in a transparent container, or can be used as a flavoring agent.

According to the invention, there is provided a transparent liquid composition comprising water, an oil-soluble ingredient, and a small amount of surfactant. The transparent liquid composition of the invention is excellent in stability, is unlikely to deteriorate the effect of the oil-soluble ingredient and is superior in safety, e.g., is low in irritability to skin.

### BEST MODE FOR CARRYING OUT THE INVENTION

The present invention will be described in more detail with reference to the following examples to which, however, the invention is not limited.

### EXAMPLE 1

### [Hair Rinse]

Ingredients (A) and (B) described below were uniformly dissolved and were mixed together with a high speed stirrer operated at 6,000 rpm for 15 minutes. The obtained opaque mixture was passed through a high pressure homogenizer (100 MPa) three times, whereby a transparent liquid composition having a transmission of 81% was obtained.

| (A) | |
|---|---|
| Cetyl octanoate | 2.0% |
| Olive oil | 1.0% |
| Fragrance | 0.1% |
| (B) | |
| Stearamidopropyl | 0.6% |
| dimethylamine phosphate | |
| Propylene glycol | 10.0% |
| Paraben | 0.3% |
| Water | 86.0% |
| Total | 100.0% |

### EXAMPLE 2

### [Hair Lotion]

Ingredients (A) and (B) described below were uniformly dissolved and were mixed together with a high speed stirrer operated at 6,000 rpm for 15 minutes. The obtained opaque mixture was passed three times through a microporous membrane having a uniform pore diameter and an average pore diameter of 0.4 μm under an increased pressure (3 MPa), whereby a transparent liquid composition having a transmission of 98% was obtained.

| (A) | |
|---|---|
| Octyl stearate | 3.0% |
| Fragrance | 0.1% |
| (B) | |
| POE (20) oleyl ether | 0.2% |
| Ethanol | 10.0% |
| Water | 86.7% |
| Total | 100.0% |

### EXAMPLE 3

### [Body Lotion]

Ingredients (A) and (B) described below were uniformly dissolved and were mixed together with a high speed stirrer operated at 6,000 rpm for 15 minutes. The obtained opaque mixture was passed five times through a ramond mixer (0.5 MPa), whereby a transparent liquid composition having a transmission of 77% was obtained.

| (A) | |
|---|---|
| Octyldodecyl lactate | 2.0% |
| L-menthol | 0.5% |
| Fragrance | 0.5% |
| (B) | |
| Decaglyceryl monolaurate | 0.05% |
| Glycerin | 5.0% |
| Paraben | 0.3% |
| Water | 91.65% |
| Total | 100.0% |

### EXAMPLE 4

### [Mouth Wash]

Ingredients (A) and (B) described below were uniformly dissolved and were mixed together with a high speed stirrer operated at 6,000 rpm for 15 minutes. The obtained opaque mixture was passed through a microporous membrane having a uniform pore diameter and an average pore diameter of 0.4 μm under an increased pressure (3 MPa), whereby a transparent liquid composition having a transmission of 99% was obtained.

| (A) | |
|---|---|
| Flavor | 0.4% |
| d-α-tocopherol acetate | 0.05% |
| (B) | |
| POE (60) hardened castor oil | 0.02% |
| Glycerin | 10.0% |
| Ethanol | 8.0% |
| Water | 81.53% |
| Total | 100.0% |

### EXAMPLE 5

### [Nutrition Supplement Drink]

Ingredients (A) and (B) described below were uniformly dissolved and were mixed together with a supersonic homogenizer (oscillating frequency 20 kHz). The obtained opaque mixture was passed five times through a ramond mixer (0.5 MPa), whereby a transparent liquid composition having a transmission of 83% was obtained.

| (A) | |
|---|---|
| d-α-tocopherol acetate | 0.3% |
| Flavor | 0.1% |
| (B) | |
| POE (60) hardened castor oil | 0.01% |
| Vitamin B₆ | 0.02% |
| Carrot extract | 1.0% |
| Benzoate | 0.2% |
| Paraben | 0.1% |
| Water | 98.27%% |
| Total | 100.0% |

### EXAMPLE 6

### [Infusion]

Ingredients (B) described below were uniformly dissolved. Ingredients (A) described below and the ingredients (B) were mixed with a supersonic homogenizer (oscillating frequency 20 kHz) and adjusted the pH to 6.0. The obtained opaque mixture was passed three times through a microporous membrane having a uniform pore diameter and an average pore diameter of 0.4 μm, whereby a transparent liquid composition having a transmission of 92% was obtained.

| (A) | |
|---|---|
| Soybean oil | 5.0% |
| Sorbitan monooleate | 0.5% |
| (B) | |
| POE (60) hardened castor oil | 1.0% |
| Water | 93.5% |
| Total | 100.0% |

### EXAMPLE 7

### [Opthalmic Solution]

Ingredients (A) and (B) described below were uniformly dissolved and were mixed together with a supersonic homogenizer (oscillating frequency 20 kHz). The obtained opaque mixture was passed three times through a high pressure homogenizer (100 MPa), whereby a transparent liquid composition having a transmission of 89% was obtained.

| (A) | |
|---|---|
| Vitamin B₁₂ | 0.01% |
| d-α-tocopherol acetate | 0.3% |
| (B) | |
| POE (60) hardened castor oil | 0.01% |
| Benzarkonium chloride | 0.05% |
| Chlorobutanol | 0.1% |
| Sodium edetate | 0.05% |
| Water | 99.48% |
| Total | 100.0% |

1. A transparent liquid composition comprising water, an oil-soluble ingredient and a surfactant.

2. The transparent liquid composition according to claim 1, which comprises 50 wt % or more of water, 30 wt % or lower of an oil-soluble ingredient and a 20 wt % or lower of a surfactant, the composition being characterized in that the content of the surfactant is less than the content of the oil-soluble ingredient.

3. The transparent liquid composition according to claim 1, which is characterized in that the content of the surfactant is ½ or lower the content of the oil-soluble ingredient.

4. The transparent liquid composition according to claim 1, which is characterized in that the surfactant is one having an HLB value of 12 or more.

5. The transparent liquid composition according to claim 1, which is characterized in that the surfactant is nonionic surfactant.

6. The transparent liquid composition according to claim 1, which is characterized in that the composition has a transmission of 70% or more.

7. A process for preparing the transparent liquid composition of claim 1, the process comprising the steps of mixing water, an oil-soluble ingredient and a surfactant to obtain an opaque mixture and further mixing the opaque mixture under an increased pressure.
